(19) United States
(12) Patent Application Publication
(10) Pub. No.: US 2021/0178012 A1
(43) Pub. Date: Jun. 17, 2021

(54) FOAM WOUND DRESSING COMPRISING AN ANTISEPTIC

(71) Applicant: Coloplast A/S, Humlebaek (DK)

(72) Inventors: Kent Hoeier Nielsen, Oelstykke (DK); Christian Aue Elbek, Virum (DK)

(21) Appl. No.: 17/254,309

(22) PCT Filed: Jul. 2, 2019
(86) PCT No.: PCT/DK2019/050213

§ 371 Date: Dec. 21, 2020

(30) Foreign Application Priority Data
Jul. 4, 2018 (DK) PA 2018 70457

(51) Int. Cl. A61L 15/42; A61L 15/44; A61L 15/20; A61L 15/26; A61K 31/444; A61K 31/14; A61L 15/48

(52) U.S. Cl. CPC A61L 15/425; A61L 15/44; A61L 15/20; A61L 15/26; A61K 31/444; A61L 2300/208; A61L 15/48; A61L 2300/404; A61L 2300/204; A61L 2420/02; A61K 31/14

(57) ABSTRACT

An open-cell foam wound dressing comprising a formulation of an amphiphilic antiseptic and particular surfactants is provided.

### TECHNICAL FIELD

The present technology relates to open-cell foams for use as wound dressings.

### BACKGROUND

Foam dressings for wound care are typically hydrophilic and absorb liquid away from a wound. Typically, such dressings are used for exuding wounds, including leg ulcers, pressure ulcers, diabetic foot ulcers, donor sites, postoperative wounds and skin abrasions.

A number of antiseptic compounds useful in wound treatment are amphiphilic, e.g. octenidine. Such compounds associate to surfaces, and have reduced mobility in a wound environment, or a hydrophilic matrix such as a foam composition.

Additionally, challenges also exist when a formulation is exposed to a sensitive wound environment. In particular, the presence of ions and other components in the wound exudate can promote the undesirable precipitation of amphiphilic components.

As an amphiphilic molecule, octenidine has shown to associate to surfaces and thereby reduce mobility in a matrix such as a foam. Early experiments documented that when octenidine is impregnated into a plain foam matrix, only a relatively low amount of octenidine was freely extractable (cf. experimental section). This strongly indicates that octenidine is attracted to the foam matrix, thereby restricting its release.

A need exists for a formulation of amphiphilic antiseptics, such as octenidine, in which the mobility of the amphiphilic antiseptic is increased in a wound environment. Additionally, the formulation should provide good solubility, mobility of the amphiphilic antiseptic and stability (i.e. lack of precipitation of the amphiphilic antiseptic). The present technology shows that the formulation of an amphiphilic antiseptic compound in a foam wound dressing can provide a major impact on the extractability, mobility and stability of said antiseptic.

### SUMMARY

An open-cell foam wound dressing is therefore provided which comprises a formulation of (a) an amphiphilic antiseptic and (b) at least one separate non-ionic surfactant or (c) at least one separate cationic surfactant or (d) at least one separate zwitterionic surfactant. The formulation can be coated on the surface of the foam wound dressing and/or incorporated into the pores of said foam wound dressing. The formulation may alternatively be comprised within the matrix of said foam wound dressing.

Methods for manufacturing such open-cell foam wound dressings are also provided.

Additional aspects of the technology are presented in the following description, the examples and the dependent claims.

### DETAILED DISCLOSURE

As set out above, an open-cell foam wound dressing comprising a formulation of (a) an amphiphilic antiseptic and (bl) at least one separate non-ionic surfactant or (b2) at least one separate cationic surfactant or (b3) at least one separate zwitterionic surfactant. The term “separate” is used to mean that the same component may not be considered as both antiseptic and surfactant, but that the formulation comprises two separate, different components.

The amphiphilic antiseptic (component a) in the formulation—being amphiphilic—has both hydrophilic and hydrophobic moieties. Examples are quaternary ammonium compounds such as benzalkonium chloride and benzethonium chloride. Biguanides such as chlorhexidine or polyhexanide (PHMB) or other cationic compounds such as octenidine and ethyl lauroyl arginate (LAE). The antiseptic is preferably octenidine. The term “amphiphilic antiseptic” includes salts thereof.

Experimental results have shown that when octenidine is impregnated into a plain foam matrix only relatively low amount of octenidine was freely extractable (see Example 1, Table 1). This strongly indicates that octenidine is being attracted to the foam matrix, which thereby restricts the release of octenidine.

The limited release of octenidine from the foam matrix can possibly be explained on the basis of the chemical structure of octenidine. Octenidine consist of two pyridines and two aliphatic tails and an aliphatic linker between the pyridinium structure. This results in an abnormal structure for a cationic detergent (see FIG. 1) and a high degree of hydrophobicity. The high degree of hydrophobicity is expected to cause the attraction to surfaces and thereby low release. Similar reasoning can be applied to other amphiphilic antiseptics.

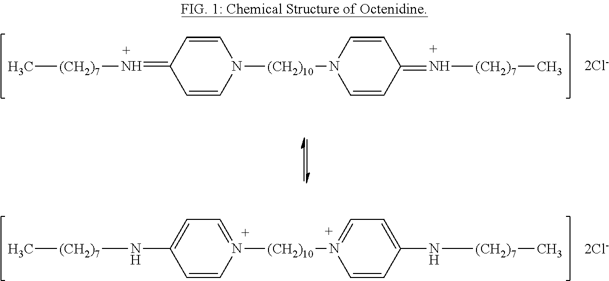

Foam Wound Dressing

The foam wound dressing may be adhesive, or non-adhesive, preferably non-adhesive. The foam wound dressing is a polymer foam e.g. a hydrophilic foam, such as a polyurethane-based foam, such as a foam of a polyether-polyurethane or polyester-polyurethane block copolymer.

Optionally, the foam wound dressing may comprise a liquid-impervious but vapor transmitting backing layer, arranged such that it faces away from the user when in use, and which prevents liquid from passing unhindered through the dressing. The backing layer may be a separate layer. A suitable material for use as a backing layer is a polyurethane film. A preferred film material is disclosed in U.S. Pat. No. 5,643,187. Alternatively, the backing layer may be formed by processing the outermost layer of the foam cells (e.g. via melting) so that a liquid barrier is provided.

The foam wound dressing may advantageously have bevelled edges, as per U.S. Pat. No. 7,875,761.

In one embodiment, the foam dressing has a density of between 100 and 400 kg/m³ such as between 120 and 300 kg/m³, or between 130 and 250 kg/m³ or even between 140 and 225 kg/m³. In a particular preferred embodiment, the density is between 150 and 200 kg/m³.

Here, density should be measured under conditions of typical use i.e. at a temperature of 20° C., air pressure of 1013 hPa, relative humidity of 40%, without compression. Under these conditions, a sample of the foam material is measured to determine the volume V and weighed to determine the mass m and the density d calculated as d=m/V.

The foam wound dressing comprises a formulation of (a) an amphiphilic antiseptic and (b1) at least one separate non-ionic surfactant or (b2) at least one separate cationic surfactant or (b3) at least one separate zwitterionic surfactant. Preferably the surfactant is (b1) at least one separate non-ionic surfactant.

By formulation is meant a formulation solution that is meant to be impregnated into the foam matrix. Following impregnation, the carrying solvent (water, ethanol or the like) is evaporated off, leaving the formulation compounds within the foam structure. Thereby, the percental concentrations within the impregnation formulation can be re-calculated into mass of compound per square (or cubic) area of foam, depending on the absorbance capacity of the given foam matrix. Example: If a foam matrix has a absorbency of 0.5mL/square centimetre and the impregnation formulation holds 0.1% amphiphilic antiseptic and 1% non-ionic surfactant. Then, the foam will be impregnated with 0.5mg amphiphilic antiseptic and 5 mg non-ionic surfactant per square centimetre. This will lead to a finished foam matrix (dried) containing 0.5 mg amphiphilic antiseptic and 5 mg non-ionic surfactant per square centimetre. For reading this document, the relation between impregnation formulation and mass per square or cubic area foam will be defined as in this section.

The formulation is suitably a solution of said components in e.g. water and/or alcohols. Suitable alcohols may be methanol or ethanol.

In one aspect, the formulation does not comprise surfactants other than the surfactants specified. In a further aspect, the formulation does not comprise antiseptics other than the antiseptic specified. In one aspect, the formulation consists of an amphiphilic antiseptic and at least one surfactant.

In one aspect, the formulation is free from inorganic salts. In particular, the formulation is free from halide salts of group I or II metals, e.g. NaCl, KCl, MgCl₂ or CaCl₂. Dissolution of the antiseptic is thereby improved.

The formulation suitably comprises between 0.001-10% w/w, preferably between 0.05-5 wt % of said amphiphilic antiseptic. The formulation suitably comprises between 0.01-10% w/w, preferably between 0.05-5 wt %, more preferably between 0.1-5 wt % of said surfactant. The dressings and formulations can show antibacterial effects even at such low concentrations ofantiseptic/surfactant. Meaning for a foam with an absorbency of 0.5 mL/cm²: 0.005-50 mg/cm² preferably between 0.25-5 mg/cm² of said amphiphilic antiseptic. The formulation suitably comprises between 0.25-50 mg/cm² w/w, preferably between 0.05-2.5 mg/cm², more preferably between 0.5-2.5 mg/cm² of said surfactant. By any deviation in exemplified absorbency (0.5 mL/cm²) the above mentioned mass contents can be corrected.In embodiments, the open-cell foam wound dressing comprises between 0.25-50 mg/cm² w/w, preferably between 0.05-2.5 mg/cm², more preferably between 0.5-2.5 mg/cm² of said surfactant. In embodiments, the open-cell foam wound dressing comprises between 0.005-50 mg/cm², preferably between 0.25-5 mg/cm², of said amphiphilic antiseptic.

The formulation may be applied to a surface of the wound dressing which is arranged to face the user when in use (i.e. the opposite face to any backing layer). Alternatively, the formulation may be applied to a surface of the wound dressing which is arranged opposite the user when in use (i.e. the opposite face to the wound contact side). Alternatively, or additionally, the formulation may be incorporated into the pores of the foam wound dressing (i.e. impregnated). Any known methods for applying the formulation into/onto the dressing may be used, such as rolling or spraying of the formulation onto a pre-formed foam wound dressing or incorporation by dipping/bathing the foam in the formulation.

In a first aspect, therefore, a method for manufacturing an open-cell foam wound dressing is provided, said method comprising:
a. Providing a formulation of (a) an amphiphilic antiseptic and (b1) at least one separate non-ionic surfactant or (b2) at least one separate cationic surfactant or (b3) at least one separate zwitterionic surfactant, said formulation additionally including a solvent;
b. Applying the formulation to a pre-formed foam wound dressing, such that the formulation becomes coated on a surface of the wound dressing and/or impregnated into the pores of the foam wound dressing.

In another aspect, a method for manufacturing an open-cell foam wound dressing is provided, said method comprising
a. Providing a formulation of (a) an amphiphilic antiseptic and (b1) at least one separate non-ionic surfactant or (b2) at least one separate cationic surfactant or (b3) at least one separate zwitterionic surfactant, said formulation optionally including a solvent;
b. blending the formulation with a foamable matrix;
c. foaming said foamable matrix together with said formulation, to provide a foam wound dressing in which said formulation is comprised within the matrix of the foam wound dressing.

As a further option, which may supplement the above options of coating/impregnating, the formulation may be comprised within the matrix of the foam wound dressing. In other words, the formulation (of antiseptic and surfactant) is blended with a foamable matrix, and then this blend is foamed. In this manner, the formulation is encapsulated within the structure of the foam, which could provide improved properties with respect to stability and release of the antiseptic.

The term “surfactant” as used herein means organic compounds that are amphiphilic, meaning they contain both hydrophobic groups and hydrophilic groups. The surfactant in the formulation is preferably non-ionic; i.e. it comprises polar hydrophilic regions which are not charged. It has been found that non-ionic surfactants provide benefits in terms of stability of the formulation and release of the antiseptic.

Alternatively, the surfactant is cationic. It has been found that cationic surfactants provide benefits in terms of stability of the formulation. Alternatively, the surfactant may be zwitterionic.

It has also been discovered that certain anionic detergents such as SDS, can interact with the antiseptic via ionic interaction and may cause precipitation and/or undesired interaction with the foam.

In one aspect, the surfactant comprises a single hydrophobic moiety, and a single hydrophilic moiety. Without being bound by theory, it is hypothesised that surfactants having one of each of such moieties can arrange optimally with the amphiphilic antiseptic.

In one aspect, the surfactant is a fatty acid monoester or fatty acid monoamide of a polyhydroxy compound. If a monoamide surfactant is used, it should be uncharged in the physiological conditions present in a wound.

According to this aspect, the fatty acid monoester or fatty acid monoamide may comprise a C2-C22 fatty acid moiety, e.g. a C4-C18 fatty acid moiety or a C6-C12 fatty acid moiety. In embodiments, the fatty acid moiety is saturated or unsaturated.

In another aspect, the surfactant is a fatty alcohol monoether of a polyhydroxy compound.

The fatty alcohol monoether may comprise a C2-C22 fatty alcohol moiety, e.g. a C4-C18 fatty alcohol moiety or a C6-C12 fatty alcohol moiety. The fatty alcohol moiety may be saturated or unsaturated.

The fatty acid moiety or said fatty alcohol moiety used herein is preferably unsaturated or saturated.

The polyhydroxy compound used as the hydrophilic moiety may be comprised of any multifunctional hydroxy- and/or amine compound (number of hydroxy groups+amine groups >=2), that may or may not be derivatized by any combination of ethylene oxide and propylene oxide. Particular polyhydroxy compounds may be selected from glycerol, sorbitan, ethoxylated sorbitan, glucose, ethylene glycol, polyethylene glycol or amine derivatives thereof.

Most preferably, the non-ionic surfactants are selected from a C6-C12 fatty alcohol monoether of glucose, or a C6-C12 fatty acid monoester of ethoxylated sorbitan. Suitable non-ionic surfactants are e.g. polysorbates (Tween) and decyl glucoside.

In a further aspect, the surfactant is a di-block copolymer (A-B), wherein one block of said copolymer (A) is hydrophobic, and the other block (B) of said copolymer is hydrophilic.

In a further aspect, the surfactant is a block copolymer and preferably a tri-block copolymer (A-B-A or B-A-B) or a di-block copolymer (A-B), wherein one block of said copolymer (A) is hydrophobic, and the other block (B) of said copolymer is hydrophilic and preferably non-ionic.

The hydrophobic block (A) may be selected from, but not limited to, polypropylene oxide, polypropylene ethylenoxide copolymers, polysiloxanes, polystyrene, polylactide, polycaprolactone and the like. Similarly, the hydrophilic block may be selected from, but not limited to, polyethylene oxide, poly(ethylene oxide co-propylene oxide), polyoxazoline, poly(vinyl pyrolidone) and the like.

In an additional aspect, the surfactant is a cationic surfactant. Such cationic surfactants comprise a cationic hydrophilic moiety and a non-ionic hydrophobic moiety. The non-ionic hydrophobic moiety of such surfactants may be a fatty acid monoester or fatty acid monoamide, such as a C2-C22 fatty acid moiety, e.g. a C4-C18 fatty acid moiety or a C6-C12 fatty acid moiety. In embodiments, the fatty acid moiety is unsaturated or saturated.

The cationic hydrophilic moiety of the cationic surfactant is typically a quaternary ammonium salt. Examples of cationic surfactants include cetrimonium bromide (CTAB), cetylpyridinium chloride (CPC), benzalkonium chloride (BAC), benzethonium chloride (BZT), dimethyldioctadecylammonium chloride and dioctadecyldimethylammonium bromide (DODAB).

In another aspect, the surfactant is a fatty alcohol monoether of a polyhydroxy compound. The fatty alcohol monoether may comprise a C2-C22 fatty alcohol.

Overall, the surfactant may have a hydrophilic-lipophilic balance (HLB) between 10 and 20 inclusive.

In embodiments, the surfactant is a zwitterionic surfactant, such as lauryl betaine (Empigen BB).

### EXAMPLES

As an amphiphilic molecule, octenidine has shown to associate to surfaces and thereby reduce mobility in a matrix such as foam. Previous studies have indicated that Octenidine did not diffuse freely in the foam matrix, indicating a high degree of interactions between octenidine and foam matrix.

To address this, we have investigated formulations to increase the mobility of octenidine by co-formulating different surface active compounds. Solubility and stability (evidenced by lack of precipitation when interacting with e.g. salts or proteins) were tested in a solution, while release was tested by infiltrating the solution into a plain foam sample, drying the foam and following carrying out release studies.

1. Octenidine in foam, no surfactant

Discs of hydrophilic polyurethane foam were impregnated by applying a known volume of octenidine-containing solution to the surface of the foam and letting it soak into the foam matrix in a liquid:foam ratio which allowed the foam to be saturated with liquid. Afterwards, the impregnated foam was dried at RT overnight.

The dried foam disc was immersed in the extraction media for 24 h and the extracted octenidine concentration was determined by UV at 285 nm

TABLE 1
These results show that when octenidine is impregnated
into a plain foam matrix only relatively low amounts
of octenidine were freely extractable.

| % Octenidine extractable | | Release % |
|---|---|---|
| after being impregnated into | | phosphate |
| plain foam w % OCT in | Release % | buffer |
| impregnation solution | water | (23 mM) |
| 0.1 w % | 5% | 7% |
| 0.5 w % | 1.5% | 13% |
| 1.0 w % | 1.5% | 20% |

2. Solubility of Octenidine with/without Surface Active Compounds.

In these experiments, octenidine dihydrochloride was dissolved in different solutions to determine the solubility with/without the presences of surface active compound (surfactant).

To investigate the interaction between the dissolved octenidine and isotonic salt concentrations (0.9%), 0.9% NaCl was co-formulated with glycerol (A4), tween (A5), or both combined (A6).

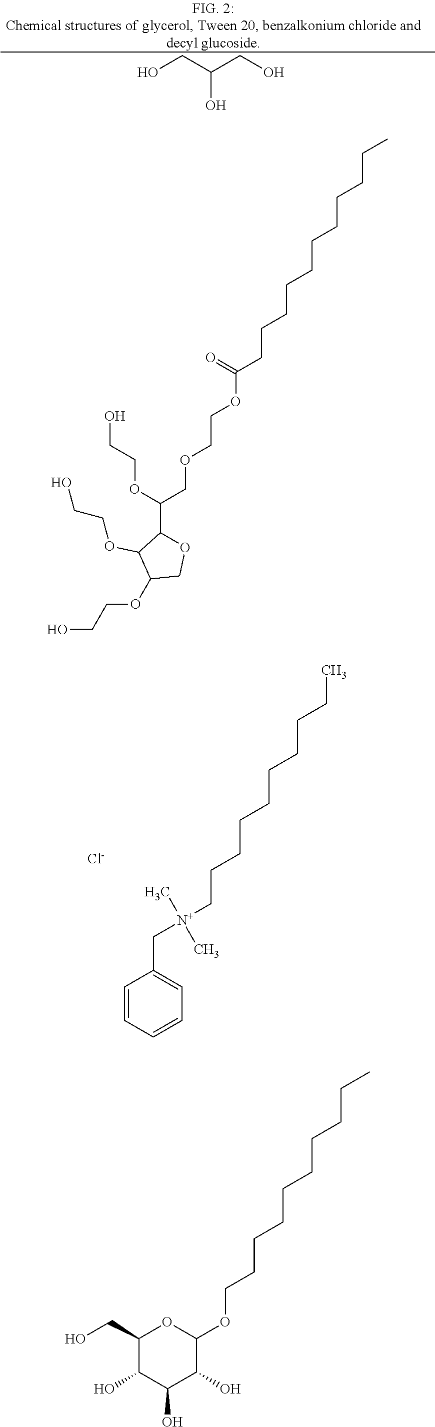 [SMILES: OCC(O)CO ; CCCCCCCCCCCC(=O)OCCOCC(OCCO)C1OCC(OCCO)C1OCCO ; [Cl-] ; CCCCCCCCCC[N+](C)(C)Cc1ccccc1 ; CCCCCCCCCCOC1O[C@H](CO)[C@@H](O)[C@H](O)[C@H]1O]

The solutions used were as follows:
A1: 3 w % Tween-20
A2: 5 w % glycerol
A3: 3 w % tween-20, 5 w % glycerol
A7. MQ water
A8: PBS buffer 23 mM
A9: 2% Benzalkonium chloride
A10: 5% Plantacare 2000 UP (50% decyl glucoside solution)

Two concentrations of Octenidine were tested: 1% and 3%
Conc. 1%: 1.00 g Octenidine+100 ml solution
Conc. 3%: 3.00 g Octenidine+100 ml solution

All solutions were prepared in conical glass bottles, sealed with plastic film at room temperature and stirred. The solutions were inspected every 15 min. and observations were recorded.

The results from the solubility tests are shown in Table 2:

TABLE 2
overview of solubilities of 1% Octenidine co-formulated
with different surfactant compounds.

| 1% | | Total time to | Dissolved after |
|---|---|---|---|
| Octenidine | pH | dissolution | 1 week at RT |
| A1 | 3.23 | 1 h | Yes |
| A2 | 5.06 | 1 h 05 min | Yes |
| A3 | 3.28 | 1 h 15 min | Yes |
| A7 | 4.99 | 1 h 20 min | Yes |
| A8 | 6.82 | 41 min | Yes |
| A9 | 6.45 | 1 h 15 min | Yes |
| A10 | 10.14 | 51 min | Yes |

All of the used solvent systems (H₂O, Glycerol, Phosphate, Tween20, Benzalkonium Cloride, and Plantacare(50% Decyl glucoside)) were able to dissolve 1%Octenidine. The solubility of 3% Octenidine was also tested and only Plantacare (solution A10) was able to fully dissolve 3% Octenidine and keep it in the dissolution without precipitation (results not shown).

Solvent systems containing salts (A4, A5, A6) did not dissolve 1% octenidine. Also, if octenidine is dissolved in respectively Tween20 or Tween20/glycerol, the same solubility/stability is indicated, while glycerol alone did not show any better solubilisation capacity than water alone. This indicates that glycerol does not have any significant effect on the solubility of octenidine, neither negative nor positive.

3. Stability of Solutions Towards Salts.

The solutions with 1% Octenidine from experiment 2 that were totally dissolved (Al, A2, A3, A7, A8, A9, A10), were tested in a new experiment. The solutions were diluted with 0.9%

NaCl to different concentrations to observe whether the Octenidine precipitated in the solution. The ratios 2:1, 1:4 and 1:10 (test solution: 0.9% NaCl) were tested and all the solutions were heated to RT (37° C.) for 1 hour. To challenge the solubility, the samples were also cooled to 4° and possible precipitation was observed.

The results are shown in Table 3:

TABLE 3
Salt stability of octenidine solutions

| 1% | | Addition of 0.9% | Addition of 0.9% | Addition of 0.9% |
|---|---|---|---|---|
| Octenidine | Temp. | NaCl solution 2:1 | NaCl solution 1:4 | NaCl solution 2:1 |
| A1 | 37° C. | No precipitation | No precipitation | No precipitation |
| | 4° C. | Precipitation | Precipitation | Precipitation |
| A2 | 37° C. | Visible precipitation | Visible precipitation | — |
| | 4° C. | — | — | — |
| A3 | 37° C. | No precipitation | No precipitation | No precipitation |
| | 4° C. | Precipitation | Precipitation | Precipitation |
| A7 | 37° C. | Visible precipitation | Visible precipitation | — |
| | 4° C. | — | — | — |
| A8 | 37° C. | No precipitation | Visible precipitation | — |
| | 4° C. | Precipitation | — | — |
| A9 | 37° C. | No precipitation | No precipitation | No precipitation |
| | 4° C. | Precipitation | Precipitation | Precipitation |
| A10 | 37° C. | No precipitation | No precipitation | No precipitation |
| | 4° C. | — | Less precipitation | Precipitation |
| | | | than other | |
| | | | solutions | |

If addition of salt is carried out after octenidine has been dissolved, the precipitating effect of NaCl is not seen at room temperature for solutions A1, A3, A9 and A10 (Table 3), indicating that an interaction between an amphiphile such as Tween20 or decyl glucoside and octenidine, protects octenidine from salt precipitation.

For all formulations except Plantacare precipitation was observed at octenidine: salt solution of 2:1 at increasing salt concentrations (1:4) slight precipitation was observed in the octentine:plantacare formulation and with even stronger precipitations at a ratio of 1:10. However, this show that decyl glucoside has the best capacity to stabilize octenidine in relation to salting out.

Overall, the 3 amphiphiles (Tween 20, benzalkonium and decyl glucoside) all dissolve 1% octenidine. But most importantly, indicated by the salt additions, they are able to stabilize octenidine in a salt-containing solution such as a wound bed and avoid precipitation upon contact with salt. Based on the temperature experiments it is indicated that decyl glucoside (Plantacare) has the best capacity to stabilize the octenidine.

4. Extraction of Octenidine from Impregnated Foams

Release profiles of octenidine in foam were studied.

Impregnation of the foam was prepared using plain Biatain (polyurethane) foam, 3 mm thick, and the foam was punched at Ø20 mm.

The 1% Octenidine solutions used for impregnations were prepared in the solubility experiments, A1, A2, A3, A7, A8, A9 and A10 as above. The volumes used for impregnation were 2 ml for Ø20 mm foam. All foam samples were placed in a fume hood overnight to dry.

Extraction Eperiment 1

The impregnated dry foam samples were cut into 4 pieces and put in a 50 ml centrifuge tube.

Samples made in triplication. As negative control a plain Biatain foam without impregnation (from the same batch) was used.

7 ml extraction solution, MQ water, Phosphate buffer 23 mm or Solution A (142 mM NaCl, 3.3 mM CaCl in MiliQwater) was added, and the sample tubes were placed on a shaking-table at 100 rpm. As will be appreciated by the skilled person, “Solution A” is an acknowledged standard solution for testing wound care devices.

Samples were taken at: 3 hours, 24 hours, 48 hours and 96 hours. Extraction samples of 500 μl were removed from the tubes at the given time-points and replaced with 500 μl new extraction solution.

The extraction samples were measured by UV at 285 nm using a micro-plate reader, and quantification of the extractions samples were carried out against a calibration curve prepared in MQ water or phosphate buffer. The calibration standards could not be dissolved in Solution A because of precipitation of Octenidine, so the extraction samples in Solution A were measured against the calibration curve obtain from standards prepared in MQ water.

Responses from the negative control samples were also calculated and used for background subtraction. The extraction solution from tubes from each time point were measured by UV as described above.

TABLE 3
Overview of the % recovery of octenidine from impregnated
foam patches using different solutions as extraction media
(using extraction test 1, above).

| | | | Solution | Phosphate |
|---|---|---|---|---|
| % recovery | | Water | A | buffer |
| 1% Oct. in 3% | 3 hours | 46.8 | 18.2 | 36.4 |
| Tween-20 | 24 hours | 48.6 | 18.8 | 42.8 |
| | 48 hours | 51.4 | 19.0 | 38.7 |
| | 96 hours | 52.6 | 21.5 | 42.7 |
| 1% Oct. in 5% | 3 hours | 39.2 | 0.4 | 16.9 |
| Glycerol | 24 hours | 42.3 | 0.5 | 20.7 |
| | 48 hours | 43.2 | 0.6 | 20.9 |
| | 96 hours | 44.7 | 0.5 | 20.5 |
| 1% Oct. in 3% | 3 hours | 44.8 | 14.8 | 30.0 |
| Tween-20 + | 24 hours | 48.9 | 20.2 | 36.6 |
| 5% Glycerol | 48 hours | 50.0 | 20.0 | 39.1 |
| | 96 hours | 48.8 | 20.7 | 41.2 |
| 1% Oct. in MQ | 3 hours | 38.3 | 0.4 | 19.5 |
| water | 24 hours | 43.2 | 0.5 | 21.8 |
| | 48 hours | 44.2 | 0.6 | 23.8 |
| | 96 hours | 45.4 | 0.5 | 24.7 |
| 1% Oct. in | 3 hours | 22.2 | 0.4 | 18.2 |
| Phosphate | 24 hours | 24.1 | 0.7 | 19.1 |
| buffer 23 mM | 48 hours | 25.9 | 0.7 | 19.3 |
| | 96 hours | 26.3 | 0.7 | 21.5 |
| 1% Oct. in 2% | 3 hours | 38.6 | 0.5 | 35.2 |
| Benzalkonium | 24 hours | 41.5 | 0.5 | 37.1 |
| chloride | 48 hours | 42.2 | 0.6 | 37.9 |
| | 96 hours | 43.4 | 0.5 | 39.6 |
| 1% Oct. in | 3 hours | 57.9 | 32.8 | 60.9 |
| 2.5% decyl | 24 hours | 55.2 | 31.8 | 67.9 |
| glucoside | 48 hours | 54.6 | 29.5 | 67.1 |
| (5% Platacare | 96 hours | 56.8 | 29.5 | 64.3 |
| 2000 UP) | | | | |

In all extractions, the release profile indicated a burst release with “full release” at first datapoint (3h). In all extraction experiments, Plantacare (A10) showed the release concentration with a max around 65% when extracted in phosphate buffer followed by ˜55% in MiliQ and ˜30% when extracted with solution A (Table 3). Between the 3 different extraction medias Tween 20 showed the second best extraction potential, while benzalkonium chloride (as an example of a cationic surfactant) showed the 3rd best extractability. In Solution A, the extractability of the benzalkonium was almost zero. This does not mean that it will necessarily be useless in a wound care application, but it does show that the examples of non-ionic (Tween, decyl glucoside) perform better and may be preferable in some applications.

Extraction Experiment 2

As in Table 3, above, all release profiles showed a burst release within the first 3 h. To understand if this represents actually available octenidine, or if it is a result of an equilibrium between dissolved and non-dissolved octenidine, the experimental setup was changed so that the foam pad was transferred to a new volume extraction media at every measuring point. Thereby the equilibrium between dissolved and non-dissolved octenidine is shifted, thereby simulating a consumption of the released octenidine as it would be expected in the wound bed.

Foam impregnated with 1% Octenidine in MQ water (A7) and 5% Plantacare (A10) were prepared described as in extraction experiment 1 with the same negative control and extraction solutions. In this experiment the foam pieces in each tube were carefully removed to a new tube containing 7 ml of fresh extraction solution at each time point.

TABLE 4
Time points: 3 hours, 24 hours, 48 hours and 72 hours.

| | | | | Phosphate |
|---|---|---|---|---|
| % recovery | | Water | Solution A | buffer |
| 1% Oct. in | 0 hours | 0.0 | 0.0 | 0.0 |
| MQ water | 3 hours | 39.8 | 0.7 | 20.2 |
| | 24 hours | 63.5 | 1.4 | 34.9 |
| | 48 hours | 77.3 | 2.0 | 46.8 |
| | 72 hours | 85.0 | 2.5 | 56.8 |
| 1% Oct. in | 0 hours | 0.0 | 0.0 | 0.0 |
| 2.5% decyl | 3 hours | 52.3 | 39.6 | 57.5 |
| glucoside | 24 hours | 69.4 | 52.1 | 76.5 |
| (5% Platacare | 48 hours | 79.5 | 54.8 | 83.5 |
| 2000 UP) | 72 hours | 85.0 | 55.4 | 85.1 |

When the balance is shifted as described, the release profile changed from burst release to a more sustained release profile. Also, the total amount released changed from around 55% to 85% for the Plantacare formulation (Table 4) illustrating that release of octenidine is a result of equilibrium between released and non-released octenidine. Solution A has a sodium concentration equal to the serum concentration and should as such better simulate the physiological conditions. In solution A, the difference between non-ionic surfactants such as

Tween 20 and Plantacare and cationic surfactants such as benzalkonium chloride is most significant.

Extranction Experiment 3

The same procedure for release test as described for experiment 1 was followed, except for the preparation of the release media. In this experiment the release media is prepared with

Plantacare in different concentrations in PBS buffer. The three different release media solutions are pH adjusted to pH 7.4 (Plantacare makes the pH increase).

| Impregnation solution | | | |
|---|---|---|---|
| | | Octenidine | |
| Solution | Surfactant Vol % | mg/mL | Release media |
| S1 | Plantacare 0.25% | 1.00 | Plantacare 0.25% in PBS |
| S3 | Plantacare 0.50% | 1.00 | Plantacare 0.50% in PBS |
| S6 | Plantacare 1.00% | 1.00 | Plantacare 1.00% in PBS |

The results, measured as recovery of octenidine in percent of the total amount of octenidine present, were as follows.

| | | Octenidine recovery (%) | | | |
|---|---|---|---|---|---|
| | | 0 hours | 24 hours | 48 hours | 72 hours |
| Sample | Release media | Average | Average | Average | Average |
| S1 | 0.25% Plantacare 2000 | 0 | 24 | 43 | 56 |
| S3 | 0.5% Plantacare 2000 | 0 | 46 | 72 | 85 |
| S6 | 1% Plantacare 2000 | 0 | 73 | 97 | 100 |

When carrying out release studies with same surfactant concentration, as used for impregnation, in the release media, significantly higher percentage release is obtained, reaching 100% for 1% Plantacare 2000 and around 56% and still rising when using 0.25% plantacare. When carrying out release without surfactants in the release media, there is a significant risk of diluting out the surfactant concentration into the release media (3×10 mL media per Ø20 mm foam disk), thereby reducing the “facilitator” for Octenidine release. By keeping the surfactant concentration constant around the octenidine molecule, better simulating the situation in the wound, the release and thereby utilization of Octenidine is becoming significantly higher. 5. Zone of Inhibition Tests

Zone of inhibition was investigated for the different formulations and at two different octenidine concentrations (0.1 and 1%). Impregnation of the foam was prepared using plain Biatain (polyurethane) foam, 3 mm thick, and the foam was punched at Ø10 mm.

The 1% Octenidine solutions used for impregnations were prepared in the solubility experiments, A1, A2, A3, A7, A8, A9 and A10 as above. The volumes used for impregnation were 0.5 ml for Ø10mm foam.

0.6% agarose plates were used. The impregnated dry samples (1% Octenidine) were pre-wetted with 400 μl MQ water before placing on the plates.

Different control samples were used for this experiment:
Positive control: standard silver (Ag) foam, Biatain
Negative control: Plain Biatain foam without PU backing film.

Control samples impregnated with solutions without Octenidine: Impregnated samples with solution A1, A2, A3, A7, A8, A9 and A10 without Octenidine added. These were prepared as per the solubility experiments above.

The foam disk (Ø10mm) was incubated with the different formulations, dried and re-wetted and placed on an agarose plate. Then, the diameter of the inhibition zone was measured after 1 day of incubation. The results are shown in Table 5 (staph. aureus) and Table 6 (pseudomonas Aeruginosa).

TABLE 5
Zone of inhibition data for staph. Aureus.
Positive control is Biatain Ag

| | Staf. Aureus | | Staf. Aureus | | Controls | |
|---|---|---|---|---|---|---|
| ZOI | 1% solutions | | 0.1% solutions | | without Oct. | |
| (average results) | Average | Stdev | Average | Stdev | Average | Stdev |
| Positive control | 7 | 0.0 | 6 | 0.0 | 5 | 0.6 |
| Negative control | 0 | 0.0 | 0 | 0.0 | 0 | 0.0 |
| Oct. in 3% | 30 | 3.5 | 29 | 3.5 | 1 | 1.2 |
| Tween-20 | | | | | | |
| Oct. in 5% | 18 | 4.2 | 15 | 3.8 | 0 | 0.0 |
| Glycerol | | | | | | |
| Oct. in 3% | 36 | 2.1 | 32 | 1.7 | 0 | 0.0 |
| Tween-20 + 5% | | | | | | |
| Glycerol | | | | | | |
| Oct. in MQ water | 18 | 3.6 | 18 | 1.0 | 0 | 0.0 |
| Oct. in Phosphate | 15 | 0.6 | 21 | 2.6 | 0 | 0.0 |
| buffer 23 mM | | | | | | |
| Oct. in 2% | 26 | 1.7 | 25 | 1.0 | 29 | 2.5 |
| Benzalkonium | | | | | | |
| chloride | | | | | | |
| Oct. in 5% | 50 | 5.1 | 53 | 2.9 | 20 | 1.0 |
| Platacare | | | | | | |
| 2000 UP | | | | | | |

TABLE 6
Zone of inhibition data for pseudomonas Aeruginosa.
Positive control is Biatain Ag

| | Pseudomonas | | Pseudomonas | | Controls | |
|---|---|---|---|---|---|---|
| ZOI | 1% solutions | | 0.1% solutions | | without Oct. | |
| (average results) | Average | Stdev | Average | Stdev | Average | Stdev |
| Positive control | 8 | 0.6 | 12 | 1.0 | 5 | 0.6 |
| Negative control | 0 | 0.0 | 0 | 0.0 | 0 | 0.0 |
| Oct. in 3% | 9 | 2.1 | 7 | 0.6 | 1 | 1.2 |
| Tween-20 | | | | | | |
| Oct. in 5% | 7 | 0.6 | 2 | 0.6 | 0 | 0.0 |
| Glycerol | | | | | | |
| Oct. in 3% | 15 | 1.0 | 10 | 1.2 | 0 | 0.0 |
| Tween-20 + 5% | | | | | | |
| Glycerol | | | | | | |
| Oct. in MQ | 7 | 1.0 | 2 | 0.0 | 0 | 0.0 |
| water | | | | | | |
| Oct. in | 6 | 0.6 | 2 | 0.0 | 0 | 0.0 |
| Phosphate | | | | | | |
| buffer 23 mM | | | | | | |
| Oct. in 2% | 9 | 1.7 | 8 | 0.6 | 29 | 2.5 |
| Benzalkonium | | | | | | |
| chloride | | | | | | |
| Oct. in 5% | 13 | 1.5 | 5 | 0.6 | 20 | 1.0 |
| Platacare | | | | | | |
| 2000 UP | | | | | | |

Previous studies (not shown) have found that pure octenidine impregnated into foam without surfactants produces small or no zones in a zone of inhibition study.

In relation to Staph. Aureus, octenidine samples show significant larger zones than for

Biatain Ag and with a clear trend that the non-ionic detergents (Tween and Decyl glycoside) increase the size of the zones. This illustrates that co-formulating with non-ionic detergents increases the mobility of octenidine in the agarose matrix. Benzalkonium is classified as an antimicrobial component in itself, which explains the signal from the negative background. Decylglucoside has in this experiment a pH above 10, most probably explaining the signal from the negative control in A10. Other experiments have shown that the antimicrobial effect of the positive control is similar at pH 10 and pH 7 (data not shown). For pseudomonas Aeruginosa the signal is not as clear mainly due to higher noise level. However, the trend is still the same; that octenidine show higher mobility when formulated with non-ionic surfactants. 6. Protein Binding and Precipitation

The purpose of this experiment is to investigate the capability of surfactant to protect Octenidine from precipitation when mixed with a protein/salt media, such as simulated wound fluid (SWF), to further understand how Octenidine and the co-formulation with detergents will respond to being released into a wound bed environment.

The results show that surfactants can significantly reduce the interaction between a protein pool and Octenidine by reducing the agglomeration of octenidine and proteins/salts. This means that the surfactants will prevent unwanted precipitation, thereby making sure that a large portion of the Octenidine is available for acting in the wound environment.

The following surfactants were tested:

| Solution | | | |
|---|---|---|---|
| no. | Surfactant: | INCI name | Batch no. |
| A | 1% Tween 20 | Polysorbate 20 | Batch #094K0052 |
| B | 1% Tween 80 | Polysorbate 80 | Lot #BCBV7863 |
| C | 1% Plantacare | Caprylyl/Capryl | lot. 17483268 |
| | 810 UP | Glucoside | |
| D | 1% Plantacare | Decyl Glucoside | lot. 0019096298 |
| | 2000 UP | | |
| E | 1% Benzalkonium | Benzalkonium | Lot #BCBV7858 |
| | chloride | chloride | |
| F | 1% Empigen BB | Lauryl Betaine | Lot #BCBQ6967 |
| G | 1% Decanesulfonate | Decane-sulfonate | Lot #BCBT6967 |
| H | 1% Plantacare | Lauryl glucoside | lot. 19090815 |
| | 1200 UP | | |
| I | Water | — | — |

The experiment was done as follows:
i) 2 ml of solution A, B, C etc., each containing 1 mg/ml Octenidine, were mixed with 2 ml SWF or water. The mix of solutions were done twice (one for each filter type).
ii) The mix of solutions were incubated for 1 hour at room temp. on a shaking table at 100 rpm.
iii) The mix of solutions were filtrated through a 0.22 μm filter. iv) The filtrated solution was diluted ten times in eluent. The Octenidine conc. should be 0.05 mg/ml (to be within detection area) if 100% was recovered after incubation and filtration.
v) Controls were prepared by diluting the formulation solution in eluent (50% Mcllvaine buffer/50% Methanol) to conc. 0.05 mg/ml (dilution x20).
vi) The samples and controls were analysed using HPLC.

The results were as follows.

| | | | Recovery | Recovery |
|---|---|---|---|---|
| Solution | | | in SWF | in water |
| no. | Surfactant: | Octenidine | (%) | (%) |
| A | 1% Tween 20 | 1 mg/ml | 99 | 100 |
| B | 1% Tween 80 | 1 mg/ml | 100 | 100 |
| C | 1% Plantacare 810 UP | 1 mg/ml | 71 | 100 |
| D | 1% Plantacare 2000 UP | 1 mg/ml | 69 | 100 |
| E | 1% Benzalkonium | 1 mg/ml | 47 | 100 |
| | chloride | | | |
| F | 1% Empigen BB | 1 mg/ml | 97 | 100 |
| G | 1% Decanesulfonate | 1 mg/ml | 7 | n.a. |
| H | 1% Plantacare 1200 UP | 1 mg/ml | 54 | 99 |
| I | Water | 1 mg/ml | 26 | 100 |

The results show that Octenidine is precipitated by mixing with protein and salt containing solutions as well as when formulated with anionic surfactants, such as decanosulfonate. However, when co-formulated with nonionic (plantacare, Tween), cationic (Benzalkonium chloride) or zwitterionic (Empigen) surfactants, Octenidine is protected against precipitation, most probably by hydrophobic-hydrophobic interaction between octenidine and detergents, scavenging the octenidine molecule from interacting with salts and/or proteins.

### CONCLUSIONS

Formulating octenidine with non-ionic or cationic surfactants, preferably non-ionic surfactants—increases the mobility and stability of the octenidine. Formulating with decyl glucoside (plantacare) resulted in the highest amount of total release octenidine with a total amount of released octenidine reaching 85% at 72 h together with an increased stability to salts. The results show that amphiphilic compounds can interact with octenidine and increase its mobility in foam and also increase stability of octenidine. Highest mobility and stability increase was seen when using decyl glucoside (Plantacare) followed by Tween 20. Glycerol did not have any effect on octenidine mobility or stability, while NaCl caused precipitation, if octenidine had not been stabilized by amphiphiles before adding salts.

Although the invention has been illustrated with reference to a number of embodiments, aspects and examples, the skilled person can combine such embodiments, aspects and examples within the scope of the appended claims.

1.-23. (canceled)

24. A wound dressing comprising a formulation of (a) an amphiphilic antiseptic and (b1) a non-ionic surfactant or (b2) a cationic surfactant or (b3) a zwitterionic surfactant; and
an open-cell foam.

25. The wound dressing according to claim 24, wherein the non-ionic surfactant comprises a fatty acid monoester or fatty acid monoamide of a polyhydroxy compound.

26. The wound dressing according to claim 25, wherein the fatty acid monoester or fatty acid monoamide comprises a C2-C22 fatty acid moiety, a C4-C18 fatty acid moiety or a C6-C12 fatty acid moiety.

27. The wound dressing according to claim 26, wherein the polyhydroxy compound comprises glycerol, sorbitan, ethoxylated sorbitan, glucose, ethylene glycol, polyethylene glycol or amine derivatives thereof.

28. The wound dressing according to claim 24, wherein the non-ionic surfactant comprises a fatty alcohol monoether of a polyhydroxy compound.

29. The wound dressing according to claim 28, wherein the fatty alcohol monoether comprises a C2-C22 fatty alcohol moiety, a C4-C18 fatty alcohol moiety or a C6-C12 fatty alcohol moiety.

30. The wound dressing according to claim 29, wherein the polyhydroxy comprises glycerol, sorbitan, ethoxylated sorbitan, glucose, ethylene glycol, polyethylene glycol or amine derivatives thereof.

31. The wound dressing according to claim 24, wherein the non-ionic surfactant comprises a tri-block copolymer (A-B-A or B-A-B) or a di-block copolymer (A-B), wherein one block of the copolymer (A) is hydrophobic, and the other block (B) of the copolymer is hydrophilic.

32. The wound dressing according to claim 31, wherein the hydrophobic block (A) comprises a polypropylene oxide, a polypropylene ethylene oxide copolymer, a polysiloxane, a polystyrene, a polylactide, or a polycaprolactone.

33. The wound dressing according to claim 31, wherein the hydrophilic block (B) comprises a polyethylene oxide, a poly(ethylene oxide co-propylene oxide), a polyoxazoline or a poly(vmyl pyrrolidone).

34. The wound dressing according to claim 24, wherein the formulation comprises a solution of the components in water and/or alcohols.

35. The wound dressing according to claim 24, wherein the amphiphilic antiseptic comprises benzalkonium chloride, benzethonium chloride, chlorhexidine, polyhexanide (PHMB), octenidine or ethyl lauroyl arginate (LAE) or salts thereof.

36. The wound dressing according to claim 24, wherein the amphiphilic antiseptic comprises octenidine or salts thereof.

37. The wound dressing according to claim 24, wherein the formulation is coated on a surface of the open-cell foam and/or incorporated into pores of the open-cell foam.

38. The wound dressing according to claim 24, wherein the non-ionic surfactant is decyl glucoside and the amphiphilic antiseptic is octenidine or salts thereof.

39. The wound dressing according to claim 37, wherein the open-cell foam comprises a polyether-polyurethane foam or a polyester-polyurethane block copolymer foam.

40. The wound dressing according to claim 24, wherein the formulation comprises between 0.001 and 10% w/w of the amphiphilic antiseptic.

41. The wound dressing according to claim 40, wherein the formulation comprises between 0.05 and 10% w/w of the surfactant.

42. A method for manufacturing a wound dressing comprising the steps:
a. providing a formulation comprising (a) an amphiphilic antiseptic and (b1) a non-ionic surfactant or (b2) a cationic surfactant, or (b3) a zwitterionic surfactant wherein the formulation further comprises an optional solvent; and
b. applying the formulation to a pre-formed open-cell foam wound dressing, such that the formulation becomes coated on a surface of the open-cell foam wound dressing and/or impregnated into the pores of the open-cell foam wound dressing.

43. A method for manufacturing a wound dressing comprising the steps:
a. providing a formulation comprising (a) an amphiphilic antiseptic and (b1) a non-ionic surfactant or (b2) a cationic surfactant, or (b3) a zwitterionic surfactant wherein the formulation further comprises an optional solvent;
b. blending the formulation with a foamable matrix; and
c. foaming the foamable matrix together with the formulation, to provide an open-cell foam wound dressing in which the formulation is comprised within the matrix of the open-cell foam wound dressing.
